(19) United States
(12) Patent Application Publication
(10) Pub. No.: US 2007/0092469 A1
(43) Pub. Date: Apr. 26, 2007

(54) Topically applied Glucosamine Sulfate and all its related, precursor, and derivative compounds significantly increases the skin's natural produciton of hyaluronic acid for the rejuvenation of healthier younger-looking skin; while PhosphatidylCholine is required to replace its deficiency caused by topical Dimethylaminoethanol (DMAE)

(76) Inventor: Eric Jacobs, Vienna, VA (US)

Correspondence Address: Eric Jacobs, P.O. Box 731, Vienna, VA, 22182, US

(21) Appl. No.: 11/527,334

(22) Filed: Sep. 27, 2006

Related U.S. Application Data
(60) Provisional application No. 60/729,947, filed on Oct. 26, 2005.

(51) Int. Cl. A61K 31/7024; A61K 31/7008; A61K 8/73

(52) U.S. Cl. 424/70.13; 514/23; 514/62

(57) ABSTRACT

A topical skin rejuvenation preparation to relieve wrinkles, increase the skin's natural production of hyaluronic acid, reverse the lack of suppleness, hydrate from within, erase spider veins, reduce varicose veins, lighten aging dark blotches (“liver spots”/Lentigos, Senile Lentigines), decrease acne, and reduce under eye puffiness includes Glucosamine (2-amino-2-deoxy-alpha-D-glucose), a hexosamine (6 carbon amino sugar), including its derivative and precursor compounds: Glucosamine Sulfate, Glucosamine Hydrochloride, Glucose-6-Phosphate, Acetyl Glucosamine, Fructose-6-phosphate, Glucosamine-6-Phosphate, to increase production of Hyaluronic acid and collagen from Glucosamine Sulfate, its precursors and derivatives and to increase skin muscle tone by Dimethylaminoethanol (DMAE) while over coming deficiency it creates in each cell's production of PhosphatidylCholine, whose deficiency damages cell membranes, as well as mitochondrial and lysosome membranes.

### CROSS REFERENCE TO RELATED APPLICATION

This application references and claims the benefit of earlier filed provisional application No. 60/729947, filed on Oct. 26, 2005.

### BACKGROUND

The present invention relates to cosmetic and topical dermatological preparations containing an effective amount of Glucosamine, Glucosamine Sulfate, Acetyl Glucosamine, and/or its derivatives and/or precursor compounds. The present invention relates in particular to cosmetic preparations having the ability to penetrate the skin where they serve as an enzyme (Hyaluronate Synthase) substrate to allow the skin to produce more hyaluronic acid, and stimulate the skin cells to produce more hyaluronic acid. Furthermore, the present invention relates in particular to cosmetic preparations having the ability to reduce skin wrinkles, increase the suppleness and soft texture of skin which is lost with aging, increase skin hydration, lighten the aging brown spots, decrease acne, shrink spider veins, reduce varicose veins, and reduce puffiness under the eyes, plus stimulate skin fibroblasts cells to produce more collagen and hyaluronic acid as well as protect these matrix compounds from oxidation through the topical action of Magnesium-L-Ascorbyl-Phosphate, plus increase subcutaneous muscle tone with the topical application of Dimethylaminoethanol (DMAE), but counter its harmful effects on reduced PhosphatidyiCholine (PC) cellular production, whose deficiency damages skin cell membranes, mitochondrial membranes and lysosome membranes, by using topical PC for its protection as well as increasing all topical nutrients trans-dermal penetration.

Furthermore, the present invention relates to cosmetic and dermatological preparations in which Glucosamine, Glucosamine Sulfate, Acetyl Glucosamine and/or its derivatives and/or precursors are used together with hyaluronidase inhibitors, such as for example inter-alpha-trypsin inhibitor.

The present invention also relates to antioxidants, and here preferably those which are used in skin care cosmetic or dermatological preparations. The invention relates particularly to cosmetic and dermatological preparations containing such antioxidant and anti-inflammatory biological agents in combination with, Glucosamine, Glucosamine Sulfate, Acetyl Glucosamine and/or its derivatives and/or its precursors. Furthermore, the present invention relates to cosmetic and dermatological skin changes, such as for aging, and here in particular skin aging produced by oxidative or degenerative processes, as well as the decrease of the skin's natural production of Glucosamine, Acetyl Glucosamine, Glucosamine Sulfate, derivative and/or precursor compounds, therefore reducing the amount of the skin's natural production of hyaluronic acid, resulting in aged, wrinkled, dry appearance, and leathery texture.

In order to prevent and help reverse the aging effects on the skin from external sources of oxidation and inflammation (sunlight and environmental) as well as internal sources of oxidation and inflammation (smoking, diet, preservatives, pesticides, chlorine, exercise) additional antioxidants and/or free-radical absorbers/scavengers and anti-inflammatories may be incorporated in the cosmetic or dermatological formulations.

Hyaluronic acid has been attempted to be used for years in topically applied cosmetic and dermatological preparations, but they are ineffective and potentially dangerous. The hyaluronic acid molecule is composed of multiple repeating of molecular pairs of “naturally occurring polyanionic, polysaccharide that consists of N-acetyl-d-glucosamine and beta-glucoronic acid.”ⁱ Thousands of these molecular pairs are attached end to end to create the final weight found in nature which is between one million and 10 million Daltons. When commercially produced, it is available from two sources:

1. Rooster combs, from which it is extracted. This results in a stiffer form of hyaluronic acid because of the cross-linking and distortion of its molecular shape. Furthermore, the chemicals used in its manufacture can remain as toxic residuals. And some people are allergic to chickens and this form of hyaluronic acid. And contamination by bacterial, fungal and viral germs are a constant danger.

2. The other source is from a bacterial fermentation process. This hyaluronic acid is more natural in its shape and consistency. But there are concerns about its purity, since the germs that produce it also excrete waste products into the brew.

However, the major hurdle that neither source can overcome is that these megasized molecules cannot penetrate into the skin. They simply and expensively stay on the surface of the skin (on top of its stratum corneum). Some companies try to produce smaller molecules to try to overcome that problem. 600,000 Dalton molecular weight hyaluronic acid is available and very expensive (thousands of dollars per kilogram) and its absorption is only a few percent. Even the more expensive 250,000 Dalton sized hyaluronic acid molecules have a very low percentage of its absorption.

Concerning the Biochemistry of Glucosamine:

“Glucosamine(2-amino-2-deoxy-alpha-D-glucose) is one of the two hexosamine sugars (6 carbon amino sugars), common in animal and human cells, (the other being galactosamine). Structurally, glucosamine is a modified glucose molecule with a NH₃ (amide) group replacing the OH (hydroxyl) group found on carbon two (C-2).ⁱⁱ

Glucosamine is found in many tissues and secretions in the body, and is the primary amino sugar substrate for the biosynthesis of the macromolecules, such as CS (chondroitin sulfate) and hyaluronic acid, which provides the framework for collagen formation. It is believed that glucosamine's role is potentiated by the presence of sulfate, which is also an essential component of proteoglycans, which provide the framework for collagen and hold water, enhancing the flexibility and resistance to compression needed to counteract physical stress. The building blocks for collagen are amino acids such as proline, glycine, and leucine; however, the building blocks for all proteoglycans are amino sugars. Glucose-6-phosphate is a building block needed as the precursor for all subsequent amino sugar synthesis. The formation of galactosamine, N-acetyl glucosamine (NAG), and CS all require Glucosamine-6-phosphate (G-6-P). Hyaluronic acid, the backbone of proteoglycans, also requires glucosamine-6-phosphate for its synthesis. Joint cartilage consists of cells embedded in a matrix of fibrous collagen within a water-proteoglycan gel. The integrity of this matrix is crucial for the biomechanical properties of the joint cartilage. The proteoglycans are larger macromolecules consisting of a protein core to which are attached multiple chains of glycosaminoglycans and oligosaccharides. Glucosamine sulfate's primary biological role in halting or reversing joint degeneration appears to be directly due to its ability to act as an essential substrate for, and to stimulate the biosynthesis of, glycoaminoglycans and hyaluronic acid backbone in the formation of the proteoglycans found in the structural matrix of joints”ⁱⁱⁱ

### SUMMARY OF THE INVENTION

It is an object of the present invention to provide cosmetic and dermatological preparations and sunscreen formulations, which serve for the prophylaxis and treatment of wrinkles, lack of suppleness, and chronic skin dehydration within the dermis and epidermis.

It is also an object of the present invention is to eliminate disadvantages of the state of the art. In particular, active ingredients or preparations containing such active ingredients should be provided, which when used may decrease spider veins, reduce varicose veins, lighten aging dark blotches from lipofucin accumulation (“liver spots”/Lentigos/Senile Lentigines), and reduce under the eye puffiness, acne, comedones, eczema, and psoriasis.

In particular, active ingredients and preparations containing such active ingredients should be provided for cosmetic and dermatological treatment and/or prophylaxis of pre-malignant skin changes such as Actinic Keratosis, as well as malignant skin cancers including Basal Cell Carcinoma, Squamous Cell Carcinoma, and Malignant Melanoma.

The present invention provides a topical skin preparation including an amount of an agent effective for prophylaxis and/or treatment of a skin change and a topical carrier. The agent includes Glucosamine, a derivative of Glucosamine, a precursor of Glucosamine and/or a combination of Glucosamine and/or a derivative of Glucosamine and/or a precursor of Glucosamine including Glucosamine Sulfate and Acetyl Glucosamine.

The present invention also provides a method for preventing, treating and/or creating a skin change including applying a topical preparation to the skin. The preparation includes an effective amount of an agent effective for preventing, treating and/or creating the skin change. The agent includes Glucosamine a derivative of Glucosamine, a precursor of Glucosamine and/or a combination of Glucosamine and/or a derivative of Glucosamine and/or a precursor of Glucosamine including Glucosamine Sulfate and Acetyl Glucosamine.

It was however surprising and not foreseeable for the expert that the use of Glucosamine, a derivative of Glucosamine, a precursor of Glucosamine and/or a combination of Glucosamine and/or a derivative of Glucosamine and/or a precursor of Glucosamine, including Glucosamine Sulfate and Acetyl Glucosamine to penetrate the skin as trans-dermally and be able to increase the skin's production of hyaluronic acid, reduce the presence of wrinkles, increase skin's suppleness, increase skin's internal hydration, reduce spider veins, reduce varicose veins, lighten aging dark blotches from lipofucin accumulation (“liver spots”/Lentigos/Senile Lentigines), and reduce under the eye puffiness, as well as with the antioxidant and anti-inflammatory compounds also prevent and reduce acne, comedones, eczema, and psoriasis, pre-cancerous and malignant skin cancers, including Basal Cell Carcinoma, Squamous Cell Carcinoma, and Malignant Melanoma, shall rectify the disadvantages of the state of the art.

### DETAILED DESCRIPTION

There are no existing products or published studies that document the use of Glucosamine Sulfate for reducing wrinkles, increasing the skin's production of hyaluronic acid and collagen, decreasing skin dryness, increasing suppleness and softness, preventing and reversing skin cancer, preventing damage from ultra violet light, functioning as an antioxidant and anti-inflammatory, for lightening (fading) aging hyperpigmentation (“lives”, “aging”) spots, strengthening vein walls, decreasing the potential for development or decreasing existing varicose veins, decreasing the size and appearance of spider veins (telangectasia), or decreasing the bruisability of the skin, and treating acne. It is not within common understanding and existing art that any of these physiological and biological changes would occur by simply applying a lotion or cream containing Glucosamine Sulfate on the skin. These claims are novel and have been substantiated by unpublished data of experiments that I have recently conducted.

The historical oral (by mouth) use of Glucosamine (Glucosamine Sulfate and/or Glucosamine Hydrochloride, which is what the following text use of “Glucosamine” refers to) “in the treatment of osteoarthritis has burgeoned recently in the USA as a result of publicity about positive clinical studies with this nutrient and to the increased availability of Glucosamine as a ‘health food’ supplement. The utility of Glucosamine for this purpose is believed to reflect a stimulatory effect on mucopolysaccharide synthesis that increases the rate of formation of extracellular cartilage matrix, as suggested by in vitro (test tube) studies and animal models.”^{iv,v,vi,vii,viii,ix,x} Furthermore, double-blind clinical studies confirmed the efficacy of Glucosamine for relieving patient pain and also for improving their mobility of osteoarthritis of the knee and hip.^{xi,xii,xiii,xiv,xv,xvi}

All of these studies used either Glucosamine Sulfate and/or Glucosamine Hydrochloride only by oral route or by intra-articular injection (directly into the affected joints).

McCarthy describes a patient who had cosmetic surgery and his doctor was amazed at the rapid healing of the surgical wound from the serendipitous oral ingestion. It was not used topically, and there was no mention of other skin benefits claimed in this scholarly article.^{xvii}

In fact McCarthy did an extensive search of the world's medical and scientific literature (“biomedical publications on wound healing”) and concluded: “administration of Glucosamine by mouth and dosage schedules of documented effectiveness in osteoarthritis will enhance hyaluronic acid (HA) synthesis in fresh wounds; an increase hyaluronic acid synthesis during the wound repair will accelerate healing and reduce the risk of scarring and related complications such as keloids, intraperitoneal (inside the abdominal cavity) adhesion and intestinal strictures.”^{xviii} Nowhere did he find any references to the oral or topical use of Glucosamine decreasing wrinkles, increasing the suppleness of the skin, increasing the hydration within the skin, erasing spider veins, shrinking or preventing varicose veins, lightening the aging dark blotches often referred to as “liver spots”, or reducing the puffiness under the eyes. Nor was there any mention made that topically applied Glucosamine in association with topically applied nutrients as referred to in this invention, will increase differentiation of pre-malignant and even malignant cells back toward or to normal.

As McCarthy clearly stated: “Glucosamine by mouth may prove to be useful not only in the treatment of surgical or traumatic wounds, but also in the management of cutaneous (skin) or gastrointestinal ulcers.”^{xix} An astute scientist like McCarthy and hundreds of other researchers, scientists, and medical doctors have also used Glucosamine in that same manner: BY MOUTH. None have used or studied its effect on the skin by topical use.

These studies on Glucosamine absorption are related to oral dosages. In the dog Glucosamine is absorbed 87%.^{xx} In humans, about 90% of Glucosamine, administered as an oral dose of Glucosamine Sulfate, is absorbed.^{xxi} N-acetylglucosamine is not well absorbed orally, since its absorption following an oral dose is all by simple diffusion and not active transport.^{xxii} There are no studies measuring the effects on the skin of transdermal absorption (through the skin) by topical application.

The only actual transdermal (through the skin) absorption study was published in January 2005: “Evaluation of the physio-chemical stability and skin permeation of Glucosamine sulfate”. These authors stated the reason for their study: “Glucosamine sulfate (GS) is known to stop the degenerative process of osteoarthritis. Because most of the GS on the market is in the oral form, an alternative formulation such as a transdermal delivery system is necessary in order to increase patient compliance.” Rubbing a cream on a body part is more time consuming and complex than simply taking a pill. Nevertheless they did their study despite the fact that it was tested in 1969 and again in 2003. These authors concluded: “results of the study (which was done on rat skin) suggested the possibility of developing Glucosamine sulfate into a transdermal delivery system.”^{xxiii}

The first article of a topical application of Glucosamine was in 1969: “Topical therapy of arthroses with Glucosamine”^{xxix,xxv} Thirty-four years later, the only other article on the topical application Glucosamine Sulfate was published. It was for a topical cream containing both Glucosamine Sulfate and Chondroitin Sulfate and camphor in which the author concluded: “Topical application of Glucosamine and Chondroitin Sulfate is effective in relieving the pain from OS (osteoarthritis) of the knee and improvement is evident within 4 weeks.” “A randomized, double-blind placebo controlled trial of a topical cream containing Glucosamine Sulfate, Chondroitin Sulfate, and camphor for osteoarthritis of the knee.”^{xxvi} What was not evident to these researchers is any feedback from any of their 63 patients (one half on the placebo and one half on this triple cream therapy), was any skin effects. Not one comment, nor in the follow-up reply to the study in 2004.^{xxvii}

Concerning decreasing the skin (“liver spot”) pigmentation for sun caused brown spots (solar lentinges), researchers attempted to use Ascorbyl Glucosamine, but had no positive effects.^{xxviii}

In 2001 a study of 53 female volunteers taking an oral supplement containing “Glucosamine, amino acids, minerals and various antioxidant compounds” versus 12 control subjects, were assessed for reduction of wrinkles and skin hydration. There was no change in epidermal (skin) hydration, but a 34% reduction in the number of visible wrinkles and also 34% reduction in the number of fine lines.^{xxix}

Based on no expectation that any transdermal absorption would remain in the skin at any potential treatment concentration, plus the fact that in all of the above there is no reference which could obviously point in the direction of the present invention, it was not possible for the expert to foresee that the Glucosamine used according to the invention including Glucosamine Sulfate, Acetyl Glucosamine and its derivatives and/or precursors, and/or any combination of these compounds or cosmetic or dermatological preparations containing them would:

act better as a wrinkle eraser

act better against skin aging and wrinkle formation

act better by increasing the skin's hyaluronic acid

act better by internally hydrating the skin

act better by reversing the skin's chronic dehydration in aging

act better by increasing the skin's suppleness

act better by eliminating spider veins

act better by eliminating acne/comedones

act better by reducing eczema

act better by reducing psoriasis

act better by preventing and reducing varicose veins

act better by fading brown skin blotches (“liver spots”)

act better by decreasing puffiness under the eyes

act better by preventing skin cancer

act better by reversing pre-malignant skin changes

act better by reversing skin cancer including Basal Cell Carcinoma, Squamous Cell Carcinoma, and Malignant Melanoma and/or

would start the regeneration process in the skin better than active ingredients and preparations of the state of the art. Furthermore, it could not have been foreseen that Glucosamine, Glucosamine Sulfate, Acetyl Glucosamine and/or its derivatives and/or precursors, and combinations, in cosmetic or dermatological preparations would have these multitude of beneficial simultaneous effects from its topical use.

A study done on volunteers assessing the topical properties led to the above-unanticipated findings. Within a few twice a day applications of this topical cosmetic preparation, visible changes were seen. And after one week the results in all subjects were remarkable. They all looked much younger. The wrinkle amount and depth was on average 50% reduced and after two weeks of twice a day applications the wrinkle number and depth was 80% less. This included crows feet, and wrinkles on the forehead, face, neck, hands, feet, elbows, knees and ankle skin areas. There was a universal youthful fullness and glow to the skin. The texture was much more supple, as in their childhood. “Liver spots” began to fade, and after 30 days of twice a day application varied from 50 to 80% less noticeable, and after more months, became even less noticeable. The fine blood vessels, generally called “spider veins”, began to disappear in one week, and after a few weeks were either completely gone in most of the volunteers, or greatly diminished in appearance. The puffiness under the eyes began to disappear in a few days, and vanished entirely in all the subjects by 10 days of twice a day application. Acne was gone in a few weeks. After a few weeks to months leg varicose veins became much less noticeable. There were no adverse reactions and all subjects demanded a continuous supply.

The cosmetic or dermatological formulations of the invention may be composed as is conventional and serve for the treatment, care and cleansing of the skin and underneath as an initial base therapeutic layer or within a make-up product in decorative cosmetics. They preferably contain 0.0001 wt. % to 30 wt. %, more preferably 0.05 wt. % to 10 wt. %, in particular 2 to 8 wt. %, based on the total weight of the agent, of Glucosamine and/or its derivatives and/or its precursors, including Glucosamine Sulfate and Acetyl Glucosamine.

For administration, the cosmetic and dermatological preparations of the invention are applied to the skin in adequate quantity in the manner conventional for cosmetics and topical dermatological skin care lotions, therapies, and/or treatments.

Cosmetic and dermatological preparations of the invention may exist in various forms. Hence, they may be, for example a solution, an anhydrous preparation, an emulsion or microemulsion of the type water-in-oil (W/O) or of the type oil-in-water (O/W), a multiple emulsion, for example of the type water-in-oil-in-water (W/O/W), a gel, a solid stick, an ointment or even an aerosol.

It is also possible and advantageous within the scope of the present invention to add Glucosamine and/or its derivatives and/or its precursors, here in particular Glucosamine Sulfate, to aqueous systems or surfactant preparations for cleansing the skin.

The use of Glucosamine for the protection of the skin from dehydration is therefore also regarded as an advantageous embodiment of the present invention, in particular this use of Glucosamine in washing formulations.

The cosmetic and dermatological preparations of the invention may contain cosmetic auxiliaries, as are used conventionally in such preparations, for example preservatives, bactericides, perfumes, substances for preventing foaming, dyestuffs, pigments which have a coloring effect, thickening agents, surfactant substances, emulsifiers, softening, moisturizing and/or moisture-retaining substances, fats, oils, waxes or other conventional constituents of a cosmetic or dermatological formulation, such as alcohols, polyols, polymers, foam stabilizers, electrolytes, organic solvents or silicone derivatives including Dimethicone.

In particular, Glucosamine, Glucosamine Sulfate, Acetyl Glucosamine, its derivatives and/or its precursors may also be combined according to the invention with antioxidants and/or free-radical absorbers and/or anti-inflammatories.

All antioxidants and anti-inflammatories which are suitable or conventional for cosmetic and/or dermatological applications may be used according to the invention as favorable anti-oxidants and anti-inflammatory agents.

The anti-oxidants are advantageously selected from the group consisting of amino acids (for example glycine, histidine, tyrosine, tryptophan) and their derivatives, imidazoles (for example urocanic acid) and their derivatives, peptides, such as D,L-carnosine, D-carnosine, L-carnosine and their derivatives (for example anserine), carotinoids, carotenes (for example alpha-carotene, beta-carotene, lycopene) and their derivatives, chlorogenic acid and its derivatives, lipoic acid and its derivatives (for example dihydrolipoic acid), aurothioglucose, propylthiouracil and other thiols (for example thioredoxin, glutathione, cysteine, cystine, cystamine and their glycosyl, N-acetyl, methyl, ethyl, propyl, amyl, butyl and lauryl, palmitoyl, oleyl, gamma-linoleyl, cholesteryl and glyceryl esters) and their salts, dilauryl thiodipropionate, distearyl thiodipropionate, thiodipropionic acid and their derivatives (esters, ethers, peptides, lipids, nucleotides, nucleosides and salts) and sulphoximine compounds (for example buthionine sulphoximines, homocysteine sulphoximine, buthionine sulphones, pentathionine sulphoximine, hexathionine sulphoximine, heptathionine sulphoximine) in very low, acceptable doses (for example μmole to .mu.moles/kg), also (metal) chelating agents (for example alpha-hydroxy fatty acids, palmitic acid, phytic acid, lactoferrin), alpha-hydroxy acids (for example citric acid, lactic acid, malic acid, mandelic acid), humic acid, colic acid, colic extracts, bilirubin, biliverdin, EDTA, EGTA and their derivatives, unsaturated fatty acids and their derivatives (for example gamma-linolenic acid, linolic acid, oleic acid), folic acid and their derivatives, ubiquinone and ubiquinol and their derivatives, vitamin C and derivatives (for example ascorbyl palmitate, Mg-ascorbyl phosphate, ascorbyl acetate), tocopherols and derivatives (for example vitamin E tocotrienol), vitamin A and derivatives (for example: retinol, retinaldehyde, retinyl esters, and vitamin A palmitate) and coniferyl benzoate of benzoin resin, rutinic acid and their derivatives, butylhydro toluene, butylhydroxy anisole, nordihydroguaiacic acid, nordihydroguaiaretic acid, trihydroxybutyrophenone, uric acid and its derivatives, mannose and its derivatives, sesamol, sesamolin, zinc and its derivatives (for example ZnO, ZnSO.sub.4), selenium and its derivatives (for example selenium methionine), stilbenes and their derivatives (for example stilbene oxide, trans-stilbene oxide) and the suitable derivatives of the invention (salts, esters, ethers, sugars, nucleotides, nucleosides, peptides and lipids) and these said active ingredients, green tea extract, EGCG (epi-gallo-catechin-gallate), grape seed extract, borage oil, gamma linolenic acid (GLA), squalane, magnesium-ascorbyl-phosphate, dimethylaminoethanol (DMAE), lecithin, phosphatidyicholine, beta sitosterol, retinol, retinaldehyde, retinyl palmitate, ginkgo biloba, extra virgin olive oil, superoxide dismutase, zinc oxide, dexpanthenol, ginseng, niacinamide (nicotinamide), ursolic acid, resveratrol, BHT, and coenzyme Q-10 (ubiquinone).

The quantity of the aforementioned anti-oxidants (one or more compounds) in the preparations is preferably 0.0001 wt. % to 30 wt. %, particularly preferably 0.05 wt. % to 20 wt. %, in particular 0.2-5 wt. %, based on the total weight of the preparation.

Provided vitamin E and/or its derivatives represent the additional antioxidant(s), it is advantageous to select their particular concentration from the range from 0.0001-20 wt. %, based on the total weight of the formulation, more preferably 0.1 to 10 wt %. and most preferably 0.3 to 2 wt. %.

Provided vitamin A or vitamin A derivatives including retinol and retinyl palmitate or carotenes or their derivatives represent the additional antioxidant(s), it is advantageous to select their particular concentrations from the range from 0.0001-10 wt. %, based on the total weight of the formulation, more preferably from 0.01 to 6 wt. % and most preferably from 0.1 to 3 wt. %.

Emulsions according to the present invention are advantageous and contain, for example the afore-mentioned fats, oils, waxes and other adipoids, and water and an emulsifier, as is used conventionally for such a type of formulation.

The lipid phase may advantageously be selected from the following substance group:

mineral oils, mineral waxes;

oils, such as triglycerides of capric or caprylic acid, also natural oils, such as for example castor oil;

fats, waxes and other natural and synthetic adipoids, preferably esters of fatty acids with alcohols of low C number, for example with isopropanol, propylene glycol or glycerine, or esters of fatty alcohols with

alkane acids of low C number or with fatty acids;

alkyl benzoates;

silicone oils, such as dimethylpolysiloxanes, diethylpolysiloxanes, diphenylpolysiloxanes, dimethicone, and mixtures thereof.

The oil phase of the emulsions, oleogels or hydrodispersions or lipodispersions within the scope of the present invention is advantageously selected from the group of esters of saturated and/or unsaturated, branched and/or unbranched alkane carboxylic acids of chain length from 3 to 30 C atoms and saturated and/or unsaturated, branched and/or unbranched alcohols of chain length from 3 to 30 C atoms, from the group of esters from aromatic carboxylic acids and saturated and/or unsaturated, branched and/or unbranched alcohols of chain length from 3 to 30 C atoms. Such ester oils may then advantageously be selected from the group isopropyl myristate, isopropyl palmitate, isopropyl stearate, isopropyl oleate, n-butyl stearate, n-hexyl laurate, n-decyloleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic, and natural mixtures of such esters, for example jojoba oil.

Furthermore, the oil phase may advantageously be selected from the group of branched and unbranched hydrocarbons and waxes, silicone oils, dialkyl ethers, the group of saturated or unsaturated, branched or unbranched alcohols, and fatty acid triglycerides, namely the triglycerine esters of saturated and/or unsaturated, branched and/or unbranched alkane carboxylic acids of chain length from 8 to 24, in particular 12-18, C atoms. The fatty acid triglycerides may advantageously be selected, for example from the group of synthetic, semi-synthetic and natural oils, for example olive oil, extra-virgin olive oil, borage oil, squalane, sunflower oil, soybean oil, peanut oil, rape-seed oil, almond oil, palm oil, coconut oil, palm kernel oil and the like.

Also any mixtures of such oil and wax components can be used advantageously within the scope of the present invention. It may also optionally be advantageous to use waxes, for example cetyl palmitate, as the single lipid component of the oil phase.

The oil phase is advantageously selected from the group 2-ethylhexyl isostearate, octyl dodecanol, isotridecyl isononanoate, isoeicosane, 2-ethylhexyl cocoate, C.sub.12-15 alkyl benzoate, capryl-capric acid triglyceride, dicaprylyl ether.

Mixtures of C.sub.12-15 alkyl benzoate and 2-ethylhexyl isostearate, mixtures of C.sub.12-15 alkyl benzoate and isotridecyl isononanoate and mixtures of C.sub.12-15 alkyl benzoate, 2-ethylhexyl isostearate and isotridecyl isononanoate are particularly advantageous.

Of the hydrocarbons, paraffin oil, squalane and squalene can be used advantageously within the scope of the present invention.

The oil phase may advantageously also contain cyclic or linear silicone oils or may consist completely of such oils, but wherein it is preferable, apart from the silicone oil or the silicone oils, to use an additional amount of other oil phase components.

Cyclomethicone (octamethylcyclotetrasiloxane) is advantageously employed as silicone oil to be used according to the invention. However, other silicone oils should also advantageously be used within the scope of the present invention, for example hexamethylcyclotrisiloxane, polydimethylsiloxane, poly(methylphenylsiloxane), dimethicone.

Mixtures of cyclomethicone and isotridecyl isononanoate, of cyclomethicone and 2-ethylhexyl isostearate, are also particularly advantageous.

The aqueous phase of the preparations of the invention contains optionally advantageously alcohols, diols or polyols of low C number, and their ethers, preferably ethanol, isopropanol, propylene glycol, glycerine, ethylene glycol, ethylene glycol monoethyl or monobutyl ether, propylene glycol monomethyl, monoethyl or monobutyl ether, diethylene glycol monomethyl or monoethyl ether and analogous products, also alcohols of low C number, for example ethanol, isopropanol, 1,2-propane diol, glycerine and in particular one or more thickening agents, which may advantageously be selected from the group silicon dioxide, aluminium silicates, polysaccharides or their derivatives, for example hyaluronic acid, xanthan gum, hydroxypropylmethylcellulose, particularly advantageously from the group of polyacrylates, in each case individually or in combination.

Mixtures of the above-mentioned solvents are used in particular. For alcoholic solvents, water may be a further constituent.

Emulsions according to the present invention are advantageous and contain, for example the afore-mentioned fats, oils, waxes and other adipoids, and water and an emulsifier, as is used conventionally for such a type of formulation.

Gels according to the present invention conventionally contain alcohols of low C number, for example ethanol, isopropanol, 1,2-propane diol, glycerine and water or an above-mentioned oil in the presence of a thickening agent, which for oily-alcoholic gels is preferably silicon dioxide or an aluminium silicate, for aqueous-alcoholic or alcoholic gels is preferably a polyacrylate.

The conventional known highly volatile, liquefied propellants, for example hydrocarbons (propane, butane, isobutane), which may be used alone or mixed with one another, are suitable as propellants for preparations which can be sprayed from aerosol containers according to the present invention: Compressed air can also advantageously be used.

Preparations according to the present invention may also advantageously contain substances which block and/or absorb UV radiation in the UVA and UVB ranges, wherein the total quantity of filter substances is, for example 0.1 wt. % to 30 wt. %, preferably 0.5 to 15 wt. %, in particular 1.0 to 10 wt. %, based on the total weight of the preparations, in order to provide cosmetic preparations which protect the skin from the entire range of ultraviolet radiation. They may also serve as sunscreen agents for the skin.

If the preparations according to the present invention contain UVA and/or UVB filter substances, they may be oil-soluble or water-soluble. According to the invention, advantageous oil-soluble UVA and/or UVB filters are, for example:

mineral oils, mineral waxes; oils, such as triglycerides of capric or caprylic acid, also natural oils, such as for example castor oil; fats, waxes and other natural and synthetic adipoids, preferably esters of fatty acids with alcohols of low C number, for example with isopropanol, propylene glycol or glycerine, or esters of fatty alcohols with alkane acids of low C number or with fatty acids; alkyl benzoates; silicone oils, such as dimethylpolysiloxanes, diethylpolysiloxanes, diphenylpolysiloxanes and mixtures thereof.

3-benzylidene camphor derivatives, preferably 3-(4-methylbenzylidene)camphor, 3-benzylidene camphor;

4-aminobenzoic acid derivatives, preferably (2-ethylhexyl) 4-(dimethylamino)benzoate, amyl 4-(dimethylamino)benzoate;

esters of cinnamic acid, preferably (2-ethylhexyl) 4-methoxycinnamate, isopentyl 4-methoxycinnamate;

esters of salicylic acid, preferably (2-ethylhexyl)salicylate, (4-isopropyl-benzyl)salicylate, homomentyl salicylate,

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzylidenemalonic acid, preferably di(2-ethylhexyl) 4-methoxybenzylidenemalonate, -2,4,6-trianilino(p-carbo-2′-ethyl-1′hexyloxy)-1,3,5-triazine.

Advantageous water-soluble UVB filters are, for example:

Ursolic acid and its derivatives; ginseng (Koren, American, Manchurian, Panax) including its extracts and isolates;

salts of 2-phenylbenzimidazole-5-sulphonic acid, such as its sodium, potassium or its triethanol-ammonium salt, and the sulphonic acid itself;

sulphonic acid derivatives of benzophenones, preferably 2-hydroxy-4-methoxybenzophenone-5-sulphonic acid and their salts;

sulphonic acid derivatives of 3-benzylidene camphor, such as for example 4-(2-oxo-3-bornylidene-methyl)benzene sulphonic acid, 2-methyl-5-(2-oxo-3-bornylidene-methyl)sulphonic acid and their salts as well as 1,4-di(2-oxo-10-sulpho-3-bornylidene-methyl)benzene and its salts (the corresponding 10-sulphato compounds, for example the corresponding sodium, potassium or triethanol;-ammonium salt), also designated as benzene-1,4-di(2-oxo-1-bornylidene-methyl)-10-sulphonic acid.

The list of the said UVB filters, which may be used in combination with the active ingredient combinations of the present invention, should of course not be limiting.

Also within the scope of the present invention is the use of a combination of Glucosamine and/or its derivatives and/or its precursors with at least one UVA and UVB filter as antioxidant or the use of a combination of Glucosamine and/or its derivatives and/or its precursors with at least one UVA and UVB filter as antioxidant in a cosmetic or dermatological preparation.

It may also be advantageous to combine Glucosamine and/or its derivatives and/or its precursors with UVA filters, which hitherto are conventionally present in cosmetic preparations. These substances are preferably derivatives of dibenzoylmethane, in particular 1-(4′-tert.butylphenyl)-3-(4′-methoxyphenyl)propane-1,3-dione and 1-phenyl-3-(4′-isopropyl-phenyl)propane-1,3-dione. These combinations or preparations which contain these combinations are also an object of the invention. The quantities used for the UVB combination may be used.

Also within the scope of the present invention is the use of a combination of Glucosamine and/or its derivatives and/or its precursors with at least one UVA filter as antioxidant or the use of a combination of the active ingredient combinations of the invention with at least one UVA filter as antioxidant in a cosmetic or dermatological preparation.

Also within the scope of the present invention is the use of a combination of Glucosamine and/or its derivatives and/or its precursors with at least one UVA filter and at least one UVB filter as antioxidant or the use of a combination of Glucosamine and/or its derivatives and/or its precursors with at least one UVA filter and at least one UVB filter as anti-oxidant in a cosmetic or dermatological preparation.

Cosmetic and dermatological preparations having an effective amount of Glucosamine and/or its derivatives and/or its precursors may also contain inorganic pigments, which are used conventionally in cosmetics to protect the skin from UV rays. They are oxides of titanium, zinc, zirconium, silicon, manganese, cerium and mixtures thereof, and modifications in which the oxides are the active agents. They are particularly preferably pigments based on titanium dioxide, including its fine (micronized) form, as well as the fine (micronized) form of zinc oxide.

These combinations of UVA filters and pigment or preparations containing this combination are also within the scope of the present invention. The quantities mentioned for the above combinations may be used.

Cosmetic preparations which are a skin-cleansing agent or shampooing agent preferably contain at least one anionic, non-ionic or amphoteric surfactant substance, or also mixtures of such substances, Glucosamine and/or its derivatives and/or its precursors in aqueous medium and auxiliaries, as are used conventionally therefore. The surfactant substance or the mixtures of these substances may be present in the shampooing agent in a concentration between 1 wt. % and 50 wt. %.

These cosmetic or dermatological preparations may also be aerosols having the auxiliaries conventionally used therefore.

Aqueous cosmetic cleansing agents of the invention or low-water or anhydrous cleansing agent concentrates intended for aqueous cleansing may contain anionic, nonionic and/or amphoteric surfactants, for example

traditional soaps, for example fatty acid salts of sodium

alkyl sulphates, alkyl ether sulphates, alkane and alkyl benzene sulphonates

sulphoacetates

sulphobetaines

sarcosinates

amidosulphobetaines

sulphosuccinates

sulphosuccinic acid semi-esters

alkyl ether carboxylates

protein-fatty acid condensates

alkylbetaines and amidobetaines

fatty acid alkanol amides

polyglycol ether derivatives.

Cosmetic preparations which are cosmetic cleansing preparations for the skin, may be present in liquid or solid form. In addition to Glucosamine, Glucosamine Sulfate, Acetyl Glucosamine and/or its derivatives and/or its precursors, they preferably contain at least one anionic, non-ionic or amphoteric surfactant substance or mixtures thereof, if required one or more electrolytes and auxiliaries, as are used conventionally therefor. The surfactant substance may be present in the cleansing preparations in a concentration between 0.001 and 99.999 wt. %, based on the total weight of the preparations.

Cosmetic preparations which are a shampooing agent, in addition to an effective amount of Glucosamine, Glucosamine Sulfate, Acetyl Glucosamine and/or its derivatives and/or its precursors, preferably contain an anionic, non-anionic or amphoteric surfactant substance or mixture thereof, optionally an electrolyte of the invention and auxiliaries, as are used conventionally therefor. The surfactant substance may be present in the shampooing agent in a concentration between 0.001 wt. % and 99.999 wt. %.

The compositions according to the present invention contain, apart from the afore-mentioned surfactants, water and optionally the additives which are conventional in cosmetics, for example perfume, thickener, dyestuffs, deodorants, antimicrobial materials, back-fatting agents, complexing and sequestering agents, pearlescent agents, plant extracts, vitamins and/other derivatives, active ingredients and the like.

The present invention also includes a cosmetic process for protecting the skin and the hair from oxidative or photooxidative processes, which is characterised in that a cosmetic agent, which contains an effective concentration of Glucosamine and/or its derivatives and/or its precursors, is applied to the skin or hair in adequate quantity.

Likewise, the present invention also includes a process for protecting cosmetic or dermatological preparations from oxidation or photooxidation, wherein these preparations, for example preparations for treating and caring for the hair are, in particular hair lacquers, shampooing agents, also make-up products, such as for example nail varnishes, lipsticks, foundations, washing and showering preparations, creams for treating or caring for skin or are all other cosmetic preparations, the constituents of which may bring with them stability problems due to oxidation or photooxidation on storage, characterised in that the cosmetic preparations have an effective amount of Glucosamine and/or its derivatives and/or its precursors and antioxidants and preservatives.

The quantity of Glucosamine, Glucosamine Sulfate, Acetyl Glucosamine and/or its derivatives and/or its precursors in these preparations is preferably 0.0001-30 wt. %, preferably 0.05-10 wt. %, in particular 0.1-6 wt. %, based on the total weight of the preparations.

Also within the scope of the present invention are processes for producing the cosmetic agents of the invention, which is characterised in that active ingredient combinations of the invention are incorporated into cosmetic and dermatological formulations in a manner known per se.

The examples below are to illustrate the present invention without restricting it. All quantity details, proportions and percentage details are, unless otherwise stated, based on the weight and the total quantity or on the total weight of the preparations.

### EXAMPLE 1

O/W lotion

| | Wt. % |
|---|---|
| Paraffin oil (DAB 9) | 8.00 |
| Petrolatum | 4.00 |
| Titanium Dioxide | 7.00 |
| Zinc Oxide | 2.00 |
| Isopropyl palmitate | 3.00 |
| Glycerine | 3.00 |
| Cetylstearyl alcohol | 2.00 |
| Butylmethoxy dibenzoylmethane | 1.00 |
| PEG-40 castor oil | 0.50 |
| Sodium cetylstearyl sulphate | 0.50 |
| Glucosamine Sulfate | 5.00 |
| DMAE(dimethylaminoethanol) | 1.50 |
| PC (phosphatidycholine) | 0.50 |
| Sodium carbomer | 0.40 |
| Alpha-tocopherol | 0.20 |
| Preservatives, dyestuffs, perfume | q.s. |
| Water | ad 100.00 |

### EXAMPLE 2

O/W cream

| | Wt. % |
|---|---|
| Paraffin oil 1 (DAB 9) | 7.00 |
| Avocado oil | 4.00 |
| Sodium lactate | 3.00 |
| Glycerine | 3.00 |
| Glyceryl monostearate | 2.00 |
| Magnesium-L-Ascorbyl-Phosphate | 0.50 |
| Titanium dioxide | 5.00 |
| Glucosamine Sulfate | 5.00 |
| Preservatives, dyestuffs, perfume | q.s. |
| Water | ad 100.00 |

### EXAMPLE 3

W/O cream

| | Wt. % |
|---|---|
| Paraffin oil (DAB 9) | 10.00 |
| Caprylic acid/capric acid triglyceride | 5.00 |
| Buxus Chinensis | 5.00 |
| Avocado Oil | 4.00 |
| PEG-40 hydrogenated castor oil | 4.00 |
| 1,2-Propylene glycol | 3.00 |
| Vaseline | 3.00 |
| Glucosamine Sulfate | 5.00 |
| DMAE(dimethylaminoethanol) | 1.50 |
| PC (phosphatidycholine) | 0.50 |
| Alpha-tocopherol | 1.00 |
| Retinol 10 CM | 1.00 |
| Alpha-tocopherol | 0.50 |
| Preservatives, dyestuffs, perfume | q.s. |
| Water | ad 100.00 |

### EXAMPLE 4

Lip care stick

| | Wt. % |
|---|---|
| Petrolatum | 40.00 |
| Magnesium-L-Ascorbyl-Phosphate | 0.50 |
| Ceresin | 8.00 |
| Hydrogenated castor oil | 4.00 |
| Beeswax | 4.00 |
| Carnauba wax | 2.00 |
| Glucosamine Sulfate | 4.00 |
| Beta-Carotene | 0.10 |
| Preservatives, dyestuffs, perfume | q.s. |
| Paraffin oil | ad 100.00 |

### EXAMPLE 5

Lip care stick

| | Wt. % |
|---|---|
| Petrolatum | 40.00 |
| Isopropyl lanolate | 10.00 |
| Beeswax, bleached | 9.00 |
| Acetylated lanolin | 4.00 |
| Carnauba wax | 4.00 |
| Glycerine | 3.00 |
| Glucosamine Sulfate | 4.0 |
| Alpha-tocopherol | 0.10 |
| Preservatives, dyestuffs, perfume | q.s. |
| Paraffin oil | ad 100.00 |

### EXAMPLE 6

Hair tonic

| | Wt. % |
|---|---|
| Ethanol | 40.00 |
| Glucosamine Sulfate | 4.0 |
| Alpha-tocopherol acetate | 0.50 |
| PEG-40 hydrogenated castor oil | 0.20 |
| Diisopropyl adipate | 0.10 |
| Preservatives, dyestuffs, perfume | q.s. |
| Water | ad 100.00 |

### EXAMPLE 7

Liposome-containing gel

| | Wt. % |
|---|---|
| Lecithin | 6.00 |
| Sorbitol | 3.00 |
| Hydrolysed collagen | 2.00 |
| Xanthan gum | 1.40 |
| Sodium citrate | 0.50 |
| Sodium PCA | 0.50 |
| Glucosamine Sulfate | 4.0 |
| Glycine | 0.20 |
| Urea | 0.20 |
| Alpha-tocopherol | 0.20 |
| Biotin | 0.08 |
| Preservatives, dyestuffs, perfume | q.s. |
| Water | ad 100.00 |

### EXAMPLE 8

Sunscreen emulsion

| | Wt. % |
|---|---|
| Titanuim Dioxide | 5.00 |
| Zinc Oxide | 2.00 |
| Castor oil | 4.00 |
| Glycerine | 3.00 |
| Octyl stearate | 3.00 |
| Magnesium-L-Ascorbyl-Phosphate | 2.50 |
| Laurylmethicone copolyol | 2.00 |
| Cyclomethicone | 2.00 |
| Cetylstearyl alcohol | 1.80 |
| Na.sub.3 HEDTA | 1.50 |
| Glycerol lanolate | 1.00 |
| Butylmethoxy dibenzoylmethane | 1.00 |
| Glucosamine Sulfate | 5.00 |
| PEG-40 hydrogenated castor oil | 0.30 |
| Sodium cetylstearyl sulphate | 0.30 |
| Acrylamide/sodium acrylate copolymer | 0.30 |
| Alpha-tocopherol | 0.20 |
| Caprylic acid/capric acid triglyceride | 0.10 |
| Preservatives, dyestuffs, perfume | q.s. |
| Water | ad 100.00 |

### EXAMPLE 9

Sunscreen emulsion

| | Wt. % |
|---|---|
| Titanuim Dioxide | 5.00 |
| Zinc Oxide | 2.00 |
| Butylmethoxy dibenzoylmethane | 4.00 |
| PEG 22-dodecyl copolymer | 3.00 |
| Paraffin oil (DAB9) | 2.00 |
| Cyclomethicone | 2.00 |
| Glucosamine Sulfate | 4.0 |
| Alpha-tocopherol acetate | 0.50 |
| Na.sub.3 HEDTA | 0.50 |
| Cetyldimethicone copolyol | 0.20 |
| Preservatives, dyestuffs, perfume | q.s. |
| Water | ad 100.00 |

### EXAMPLE 10

Sunscreen emulsion

| | Wt. % |
|---|---|
| Titanuim Dioxide | 5.00 |
| Zinc Oxide | 2.00 |
| Castor oil | 4.00 |
| Octyl stearate | 3.00 |
| Glycerine | 3.00 |
| Cyclomethicone | 2.00 |
| Laurylmethicone copolyol | 2.00 |
| Cetylstearyl alcohol | 1.70 |
| Na.sub.3 HEDTA | 1.50 |
| Glycerol lanolate | 1.00 |
| Butylmethoxy dibenzoylmethane | 1.00 |
| Alpha-tocopherol acetate | 1.00 |
| Glucosamine Sulfate | 5.00 |
| PEG-40 hydrogenated castor oil | 0.40 |
| Sodium cetylstearyl sulphate | 0.30 |
| Acrylamide/sodium acrylate copolymer | 0.30 |
| Hydroxypropylmethylcellulose | 0.30 |
| Caprylic acid/capric acid triglyceride | 0.10 |
| Preservatives, dyestuffs, perfume | q.s. |
| Water | ad 100.00 |

### EXAMPLE 11

| Gel | Wt. % |
|---|---|
| Triethanolamine | 3.00 |
| Carbopol 934 P | 2.00 |
| Hydrolysed (non-bovine) collagen | 2.00 |
| Glycerine | 2.00 |
| Titanium Dioxide | 5.00 |
| Zinc Oxide | 2.00 |
| Sodium PCA | 0.50 |
| Glucosamine Sulfate | 4.0 |
| Alpha-tocopherol acetate | 0.20 |
| Preservatives, dyestuffs, perfume | q.s. |
| Water | ad 100.00 |

### EXAMPLE 12

| Spray formulation | Wt. % |
|---|---|
| Ethanol | 30.00 |
| Glucosamine Sulfate | 5.00 |
| Alpha-tocopherol | 0.20 |
| Magnesium-L-Ascorbyl-Phosphate | 2.50 |
| DMAE (dimethylaminoethanol) | 1.50 |
| PC (phosphatidycholine) | 0.50 |
| Preservatives, dyestuffs, perfume | q.s. |
| Propane/butane 25/75 | ad 100.00 |

In examining the prior art we found our patent to be unique:
1. U.S. Pat. No. 5,391,373 (Mausner) is distinguished by its failure to use an adequate amount of Glucosamine in order to achieve the substrate concentration for the enzyme Hyaluronate Synthase to produce a sufficient quantity and concentration of Hyaluronic Acid necessary to achieve any meaningful change in the texture of the skin, including its ability to hold onto sufficient water to increase internal hydration. Their patent claims: “1. (d) about 0.001% to 0.1% of a carbohydrate-based complex comprising dextran, glycine, and Glucosamine;”. Furthermore, in their “Summary” they state: “A preferred skin cream composition of the present invention comprises: (4) a carbohydrate-based complex of dextran comprises from about 70% to about 90% of the carbohydrate-based complex, the glycine comprises from about 10% to about 20% of the carbohydrate-based complex, and the Glucosamine comprises from about 5% to about 15% of the carbohydrate-based complex.”
Therefore, with their patent formulation of Glucosamine in a mixture of compounds at “5% to about 15% of the carbohydrate-based complex”, when multiplied by the 0.001% to 0.1%, the actual range of Glucosamine, what they really claimed for Glucosamine is from 0.1 % (0.001) times 15% (0.15) equals 0.00015(0.015%) =0.015 grams per 100 milliliters=only 15 milligrams per 100 milliliters as its maximum possible concentration which is totally not effective in increasing the intra-dermal concentration of Hyaluronic Acid by its topical application. Their minimum range amount claimed is calculated as: 0.001% (0.00001) times 5% (0.05%) equals 0.0000005, which is 0.00005% and equals 5 one-hundred-thousandths of a gram per 100 milliliters, and is totally inconsequential for having any positive effect. Both extremes of their range, maximum or minimum are not effective and they're not relevant to creating intra-dermal Hyaluronic Acid. Furthermore, Glucosamine Sulfate is the most effective form of this critical organic compound.
2. U.S. Pat. No. 5,141,964 (Noel) claims in #1: “ . . . to moisturize and improve the surface appearance of skin”. And in “The Invention” text has the Glucosamine in “a cosmetic base” whose total function is “ . . . to moisturize and improve the surface condition of the epidermis.” There is no claim for increasing the skin's production of Hyaluronic Acid. Furthermore, the layer of the skin which produces the Hyaluronic Acid is the dermis, which is much deeper and thicker than the surface epidermis layer. This patent confirms that no valid claim for topical Glucosamine existed for increasing the intra-dermal concentration of Hyaluronic Acid, as was known in the prior art.
3. U.S. Pat. No. 5,866,142 (Riordan) specifically claims only a “form of Glucosamine” in claims #1, 10, and 21, and that is the only form of Glucosamine specified is just N-acetyl-D-Glucosamine as noted in claims #7, 17, 28, 31, 38, and 42. Furthermore, in the text under “The Summary of the Invention” it states: “4. Providing the rate limiting substrates for the production of Hyaluronic Acid by the skin cells;” and in the following text, in numerous locations specifies that the form is to be “N-acetyl-D-Glucosamine” and that: “N-acetyl-D-Glucosamine is known to be a rate-limiting factor in the Hyaluronic Acid production by living cells.”
There are a number of rate-limiting factors in the cellular production of Hyaluronic Acid. Unfortunately, N-acetyl-D-Glucosamine is not well absorbed into the body, and therefore is not the substrate of cellular choice for the production of Hyaluronic Acid, as at present invention discusses in biochemical and physiological detail. Glucosamine, and in particular, Glucosamine Sulfate is much better absorbed into the body via the skin, and its effect on markedly enhancing the production of the skin's Hyaluronic Acid has been demonstrated for the present invention in live clinical human experiments including with applications to one side of the face, neck and hands with the subjects other side being the control using the base (placebo) lotion, with only the inventor of knowing which side contained the Glucosamine Sulfate, which was at a 5% concentration.
In the Riordan patent under the “Detailed Description of the Invention” it teaches that: “The preferred concentration of N-acetyl-D-Glucosamine . . . , and preferably 0.1% by weight.” Not only is that form of Glucosamine very poorly absorbed through the skin, but the patent's “most preferably 0.1%” is worthless as serving as any effective substrate for the cells ability to produce any adequate amount of Hyaluronic Acid to serve the purpose of the invention: reducing wrinkles and improving the appearance of the skin. Perhaps it can achieve those laudable goals through its claimed use of exfoliation through the use of histadine and/or its chelation with taurine and/or EDTA as the compound to remove calcium allegedly damaging the elastic fibers. But there is no claim for or use of Glucosamine, nor for Glucosamine Sulfate, which is the best and most absorbed substrate for the skin's enzyme, Hyaluronate Synthase, to make adequate amounts of Hyaluronic Acid.
4. U.S. Pat. No. 6,413,525 (Mammone) uses only N-acetyl-D-Glucosamine for the only claim of “exfoliation of the skin”.
5. U.S. Pat. No. 5,728,661 (Petit) claims only a method of “forming N-acetyl-D-Glucosamine from Glucosamine hydrochloride”. There is no claim for Glucosamine used topically to penetrate the skin and serve as a substrate for the skin cells to produce Hyaluronic Acid. There is no claim for Glucosamine Sulfate. It discusses a “method of providing a surface-active property to a composition”.
6. U.S. Pat. No. 6,028,118 (Dupont) is specific for using shark cartilage extracts, and methods for such extraction, for the skin “for improving mammalian skin barrier function which translates into more resistance to transdermal water loss”, for processes for shark cartilage extraction and preparation, and for its uses and disease states. There are no claims for Glucosamine being applied topically, and no claims for topical Glucosamine or Glucosamine Sulfate serving as a substrate and stimulant for the dermal layer of the skin to produce Hyaluronic Acid.

### STATEMENT REGARDING FEDERALLY SPONSORED RESEARCH

Not Applicable

What is claimed is:

1. A method for creating a skin change comprising applying a topical preparation to the skin, the preparation comprising an amount of an agent effective for creating the skin change, the agent comprising Glucosamine, and/or Glucosamine Sulfate, and/or Glucosamine Hydrochloride, and/or Glucose-6-phosphate, and /or Fructose-6-phosphate, and/or Acetyl Glucosamine, and/or Glucosamine-6-Phosphate, a derivative of these, a precursor of any of these or a combination of any of these and the derivative and/or precursor of any of these.

2. The method as recited in claim 1 wherein the skin change includes preventing and reversing aging of the skin.

3. The method as recited in claim 1 wherein the skin change includes preventing and reversing wrinkling of the skin.

4. The method as recited in claim 1 wherein the skin change includes preventing and reversing damage of the skin from ultraviolet light.

5. The method as recited in claim 1 wherein the skin change includes preventing and reversing an oxidative process and inflammation.

6. The method as recited in claim 1 wherein the skin change includes preventing and reversing degenerative processes.

7. The method as recited in claim 1 wherein the skin change includes preventing, treating and resolving acne, comedones, eczema, and psoriasis.

8. The method as recited in claim 1 wherein the skin change includes actinic keratosis, pre-cancerous changes, and all forms of skin cancer including basal cell carcinoma, squamous cell carcinoma, and, malignant melanoma.

9. The method as recited in claim 1 wherein the skin change includes preventing and reversing loss of suppleness.

10. The method as recited in claim 1 wherein the skin change includes preventing and reversing the decrease in the skin's Hyaluronic Acid.

11. The method as recited in claim 1 wherein the skin change includes preventing and reversing drying of the skin.

12. The method as recited in claim 1 wherein the skin change includes brown blotches from lipofucin accumulation (“liver spots”/Lentigos/Senile Lentigines).

13. The method as recited in claim 1 wherein the skin change includes preventing and reversing reduction in spider veins (telangectasia), decrease in varicose veins, and decreases bruisability by its strengthening ability of the walls of these dilated blood vessels.

14. The method as recited in claim 1 wherein the skin change includes preventing and reducing puffiness under the eyes.

15. The method as recited in claim 1 wherein the agent acts as a nutrient substrate for the skin to manufacture Hyaluronic Acid.

16. The method as recited in claim 1 wherein the agent acts as an antioxidant.

17. The method as recited in claim 1 wherein the agent acts as a free radical scavenger.

18. The method as recited in claim 1 wherein the agent supports the mobilization and growth of skin cells so as to promote regeneration of the skin cells.

19. The method as recited in claim 1 wherein the precursor or derivative of Glucosamine is Glucosamine Sulfate.

20. The method as recited in claim 1 wherein the precursor or derivative of Glucosamine is Glucosamine Hydrochloride.

21. The method as recited in claim 1 wherein the precursor or derivative of Glucosamine is Glucose-6-Phosphate.

22. The method as recited in claim 1 wherein the precursororr derivative of Glucosamine is Fructose-6-Phosphate.

23. The method as recited in claim 1 wherein the precursor or derivative of Glucosamine is Glucosamine-6-Phosphate.

24. The method as recited in claim 1 wherein the precursor or derivative of Glucosamine is Acetyl Glucosamine.

25. The method as recited in claim 1 wherein Glucosamine, and/or Glucosamine Sulfate, Glucosamine Hydrochloride, Glucose-6-Phosphate, Acetyl Glucosamine, Fructose-6-Phosphate, and/or Glucosamine-6-Phosphate, a derivative of these, a precursor of any of these or a combination of these and the derivative and/or precursor of any of these has a concentration of from about 0.01 to about 50% by weight of the preparation.

26. The method as recited in claim 1 wherein Glucosamine and/or GlucosamineSulfate, Glucosamine Hydrochloride, Glucose-6-Phosphate, Acetyl Glucosamine, Fructose-6-Phosphate, and/or Glucosamine-6-Phosphate, a derivative of these, a precursor of any of these or a combination of these and the derivative and/or precursor of any of these has a concentration of from about 0.1 to about 15% by weight of the preparation.

27. The method as recited in claim 1 wherein the Glucosamine and/or Glucosamine Sulfate, Glucosamine Hydrochloride, Glucose-6-Phosphate, Acetyl Glucosamine, Fructose-6-Phosphate, and/or Glucosamine-6-Phosphate, a derivative of these, a precursor of any of these or a combination of these and the derivative and/or precursor of any of these has a concentration of these is about 1.0 to about 7.0% by weight of the preparation.

28. The method as recited in claim 1 wherein the preparation is a cosmetic preparation.

29. The method as recited in claim 1 wherein the preparation is a dermatological preparation.

30. A method for creating a skin change comprising: providing a person in need of treatment of the skin change, the skin change being responsive to an application of Glucosamine and/or Glucosamine Sulfate, Glucosamine Hydrochloride, Glucose-6-Phosphate, Fructose-6-Phosphate, Acetyl Glucosamine and/or Glucosamine-6-Phosphate, a derivative of these, a precursor of any of these or a combination of these and the derivative and/or precursor of any of these.; and
applying an amount of the Glucosamine and/or Glucosamine Sulfate, Glucosamine Hydrochloride, Glucose-6-Phosphate, Acetyl Glucosamine, Fructose-6-Phosphate, and/or Glucosamine-6-Phosphate, a derivative of these, a precursor of any of these or a combination of these and the derivative and/or precursor of any of these, effective for creating the skin change.

31. The method as recited in claim 30 wherein the topical skin preparation further comprises at least one compound selected from the group consisting of vitamin A and its precursors, vitamin E and its precursors and related Tocopherols and Tocotrienols, green tea extract, EGCG (epi-gallo-catechin-gallate), grape seed extract, borage oil, gamma linolenic acid (GLA), squalane, magnesium-ascorbyl-phosphate, dimethylaminoethanol (DMAE), lecithin, phosphatidyicholine, beta sitosterol, retinol, retinyl palmitate, ginkgo biloba, extra virgin olive oil, superoxide dismutase, zinc oxide, titanium dioxide, dexpanthenol, ginseng, vitamin D (cholecalciferol) niacinamide (nicotinamide), ursolic acid, resveratrol, inter-alpha-trypsin inhibitor, BHT and coenzyme Q-10 (ubiquinone).

32. What is claimed is the combination of Dimethylaminoethanol (DMAE) and PhosphatidylCholine (PC) where the negative effect of DMAE that interferes with the body's natural production of PC by the skin cells, skin cell membranes, mitochondrial membranes, and lysosomal membranes is augmented by the topical application of PC which penetrates into the skin to make up for the deficiency and harm created by the topical use of DMAE without PC.

33. As claimed in 32 the concentration of DMAE is from 0.0001% to 50% (where percent means grams per 100 milliliter for lotion and grams per 100 grams weight for creams), with a better concentration of 0.1% to 15%, and the best concentration from 0.5% to 4%.

34. As claimed in 32 the concentration of PhosphatidylCholine is 0.01% to 30%, with a better concentration of 0.1% to 15% and the best concentration of 0.5 to 10%.

35. As claimed in 32 the combination of Dimethylaminoethanol (DMAE) and PhosphatidylCholine (PC) topically is unique and needed because DMAE is an inhibitor of the body's (skin's) production of PhosphatidylCholine. Dimethylaminoethanol (DMAE) is related to the B Vitamin Choline, and happens to be a precursor (substance that is used to make another substance) of the neurotransmitter (nerve-to-nerve stimulating compound) acetylcholine. New medical evidence also had shown that acetycholine functions as a ubiquitous cytokine-like molecule that has the ability to regulate cellular processes including proliferation (cellular division) and differentiation (change from one cell type into a more defined form). This specific combination and for this healthful and protective purpose for the skin is not known in the prior art.

36. What is claimed is the combined topical application of both Magnesium-L-Ascorbyl-Phosphate and Glucosamine Sulfate.

37. As claimed in 36, the Magnesium-L-Ascorbyl-Phosphate penetrates the skin and is a “reservoir” source for Vitamin C (Ascorbic Acid) which stimulates the fibroblast skin cells to produce more Hyaluronic Acid and the Glucosamine Sulfate serves as the substrate for the enzyme Hyalurate Synthase to use the Glucosamine Sulfate to manufacture more Hyaluronic Acid and to stabilize and protect the Hyaluronic Acid produced from free radical (oxidant) damage and degradation.

38. As claimed in 36, the Magnesium-L-Ascorbyl-Phosphate serves as the intra-dermal “reservoir” for Vitamin C (Ascorbic Acid) and stimulates the skin fibroblast cells to utilize the Glucosamine Sulfate to increase the production of Collagen and to stabilize the Collagen produced and protect it from free radical (oxidant) damage and degradation.

39. Referring to claim 36, the concentration of the Magnesium-L-Ascorbyl-Phosphate is from 0.001% to 50% (where percent means grams per 100 milliliters for lotion's and grams per 100 grams weight for creams), with a better concentration of 0.1% to 15% and the best concentration from 0.3% to 6%.

40. Referring to claim 36 the concentration of Glucosamine Sulfate is 0.01% to 50%, with a better concentration of 0.1% to 15%, and the best concentration is 1% to 7%.
